(19) United States
(12) Patent Application Publication
(10) Pub. No.: US 2015/0309048 A1
(43) Pub. Date: Oct. 29, 2015

(54) METHODS AND KITS FOR DETECTING ANTIGEN-INDUCED MEMORY CD8+ T CELLS

(71) Applicants: INSERM (ISNTITUT NATIONAL DE LA SANTE ET DE LA RECHERCHE MÉDICALE), Paris (FR); UNIVERSITÉ CLAUDE BERNARD - LYON 1, Villeurbanne Cedex (FR); CENTRE NATIONAL DE LA RECHERCHE SCIENTIFIQUE (CNRS), Paris (FR)

(72) Inventors: Jacqueline MARVEL, Lyon (FR); Sophia DJEBALI, Lyon (FR); Morgan GRAU, Lyon (FR); Julien MAFILLE, Lyon (FR); Yann LEVERRIER, Lyon (FR)

(21) Appl. No.: 14/653,374

(22) PCT Filed: Dec. 20, 2013
(86) PCT No.: PCT/EP2013/077767

§ 371 Date: Jun. 18, 2015

(30) Foreign Application Priority Data
Dec. 21, 2012 (EP) 12306646.6

(51) Int. Cl. G01N 33/68

(52) U.S. Cl. CPC G01N 33/6863; G01N 2333/523

(57) ABSTRACT

The present invention relates to methods and kits for detecting antigen-induced memory CD8+ T cells. In particular, the invention relates to a method for determining the presence of a population of antigen-induced memory CD8+ T cells in a sample, comprising i) isolating the population of the CD8+ T cells from the sample and ii) detecting in said population the expression of CCL5 or NKG2D and iii) concluding that at least one population of antigen-induced memory CD8+ T cells is present in a sample when CCL5 or NKG2D is detected as step ii).

### FIELD OF THE INVENTION

The present invention relates to methods and kits for detecting antigen-induced memory CD8+ T cells.

### BACKGROUND OF THE INVENTION

One hallmark of the immune system is its capability to respond faster to a second exposure to the same antigen: a phenomenon called memory. This property is the basis for protective vaccination against infectious diseases or tumors. Immunological memory is based on antigen-specific memory B and T cells that are antigen specific cells that will persist long after infection has resolved.

The naive T cell compartment is composed of a huge diversity of naive CD8⁺ T cells bearing antigen specific receptors (TCR, for T Cell Receptor). During the primary response (first encounter with an antigen), the rare naive CD8⁺ T cells that are antigen specific are activated, expand and differentiate into effector cells within the first 7 to 10 days of the primary response¹. CD8⁺ T cell activation is a complex process which depends on the recognition of peptide (derived from pathogen-antigens) loaded onto MHC class I molecules at the surface of professional Antigen Presenting Cells (APC) such as dendritic cells (DC). Three signals are required for optimal CD8⁺ T cell activation: TCR recognition of peptide MHC class I complex (signal 1), T cell's CD28 engagement by costimulatory molecules expressed at the surface of APC following the recognition of danger signals such as PAMPS (Pathogen Associated Molecular Pattern) (signal 2) and inflammatory cytokines (signal 3)¹. After their activation, naive cells differentiate into effector CD8⁺ T cells. Effector cells destroy pathogen-infected cells through cytotoxicity notably mediated by the release of cytotoxic granules containing perforin and granzymes. Effector CD8⁺ T cells also produce cytokines such as IFNγ and TNFα, which are key anti-viral players¹. Following pathogen elimination, the majority of effector CD8⁺ T cells undergo apoptosis (contraction phase) while some of them will differentiate into memory cells^{1, 2}.

Memory cells can be distinguished from naive cells based on their expression of a number of surface markers such as CD44 and CXCR3. Functionally, memory CD8⁺ T cells are resting cells but are hyper-responsive, allowing them to rapidly re-express effector functions upon TCR re-stimulation^{3 4}. The CD8⁺ T cell memory compartment is heterogeneous. The memory compartment is composed of antigen-induced memory cells, for example the ones that have been generated after pathogen encounter (pathogen-induced memory CD8⁺ T cells) and that represent true memory cells. However the memory compartment is also composed of cytokine-induced memory phenotype cells (innate and Homeostatic Proliferation (HP) memory-phenotype CD8⁺ T cells). Among the antigen-induced memory T cells, two subsets have been well characterized: T_{CM} (T Central Memory cells) and T_{EM} (T Effector Memory cells). These cells are generated following infection or tumor rejection. These subsets both express CD44 and CXCR3 but differ in their homing and effector capacities. T_{CM} home to secondary lymphoid organs, express IL-2 and vigorously proliferate upon antigen re-encounter whereas T_{EM} gain access to peripheral tissues, display potent cytotoxicity but proliferate poorly⁵. Another antigen-induced memory subset is the T inflammatory memory subset (T_{IM}). These cells, described in the team, are generated by priming F5 TCR transgenic mice with antigen under sterile inflammatory conditions (i.e. in the absence of pathogen-derived signals)⁶. T_{IM} have been shown to be involved in the recall antigen-specific contact-hypersensitivity reactions. T_{IM} are arrested at a relatively early memory-stage and are able to further differentiate into T_{CM} or T_{EM} memory CD8⁺ T cells upon antigenic re-stimulation by PAMP-matured DC⁶ or after viral infection (Jubin V, Ventre E, Leverrier Y, Djebali S, Mayol K, Tomkowiak M, Mafille J, Teixeira M, Teoh D Y, Lina B, Walzer T, Arpin C, Marvel J. T inflammatory memory CD8 T cells participate to antiviral response and generate secondary memory cells with an advantage in XCL1 production. Immunol Res. 2012 June; 52(3):284-93.).

It is critical to be able to evaluate the magnitude and the kinetic of memory CD8⁺ T cell generation for understanding the regulation of the immune responses but also to be able to evaluate vaccine candidates. One way to do so is to track and analyse antigen-specific CD8⁺ T cells, but that requires the knowledge of the specific epitopes, i.e. antigen specificities, that will dominate the immune response. However, the full spectrum of antigenic epitopes harbored by a pathogen or a vaccine is often partially unknown. One other way would be to follow the whole antigen-induced population. Unfortunately, as described above, memory compartment is composed of antigen-induced and cytokine-induced memory cells and the phenotypic markers used to detect these memory cells (CD44, CXCR3, IFNγ) do not allow to discriminate between them. The identification of a biomarker that would be specific for antigen-induced memory cells would allow monitoring more closely the generation of memory CD8⁺ T cells in response to an infection or vaccination.

CCL5 is one of the most up-regulated genes at the mRNA level in pathogen-induced or T_{IM} memory cells compare to naive CD8⁺ T cells. Ccl5 gene encodes a 68 amino acid chemokine, previously called RANTES (Regulated upon Activation Normal T cells Expressed and Secreted). A broad range of both immune and non-immune cells produce it¹⁵. CCL5 preferentially binds its seven transmembrane G protein-coupled receptor CCR5, although it can also binds CCR1 and CCR3. CCL5 is a chemoattractant that induces chemotaxis of target cells through the activation of G protein dependent pathways¹⁶. CCL5 is a pro-inflammatory chemokine and high production of CCL5 is frequently associated with inflammatory disorders, like asthma or arthritis¹⁵. A broad range of cells are sensitive to CCL5-induced chemotaxis and are therefore attracted to the sites of inflammation by CCL5. CCL5 has been demonstrated to play an important role during immune responses against viral infections¹⁷ but the precise role of the CCL5 produced by the memory CD8⁺ T cells is still unclear.

### SUMMARY OF THE INVENTION

The present invention relates to a method for determining the presence of a population of antigen-induced memory CD8+ T cells in a sample, comprising i) isolating the population of the CD8+ T cells from the sample and ii) detecting in said population the expression of CCL5 or NKG2D and iii) concluding that at least one population of antigen-induced memory CD8+ T cells is present in a sample when CCL5 or NKG2D is detected as step ii).

### DETAILED DESCRIPTION OF THE INVENTION

There is currently no phenotypic marker enabling the discrimination of cytokine-versus antigen-induced memory CD8⁺ T cells. Results obtained by the inventors have highlighted a potential biomarker for antigen-induced CD8⁺ memory cells: the chemokine CCL5. Thus, the inventors have measured CCL5 expression by different memory CD8⁺ T cells populations at the protein and mRNA levels. CCL5 protein is expressed by antigen-induced memory CD8⁺ T cells but not by cytokine-induced memory cells. Antigen-induced memory cells express high levels of CCL5 mRNA whereas homeostatic proliferation memory-phenotype cells do not express significant levels of ccl5 transcripts. The results indicate that the chemokine CCL5 is a biomarker for antigen-induced memory CD8⁺ T cells that could allow tracking of truly antigen-experienced memory cells in the follow up of immune responses, such as vaccination protocols. The inventors also demonstrated that expression of CCL5 is correlated with the expression of NKG2D at the surface of antigen-induced memory CD8⁺ T cells in the mouse and thus concluded that NKG2D can be used for determining the presence of a population of antigen-induced memory CD8+ T cells in a murine sample.

Accordingly the present invention relates to a method, for determining the presence of a population of antigen-induced memory CD8+ T cells in a sample, comprising i) isolating the population of the CD8+ T cells from the sample and ii) detecting in said population the expression of CCL5 or NKG2D and iii) concluding that at least one population of antigen-induced memory CD8+ T cells is present in a sample when CCL5 or NKG2D is detected as step ii).

Optionally the method of the invention may further comprise a step iv) consisting of isolating the population of antigen-induced memory CD8+ T cells detected at step iii).

As used herein the term “CD8+ T cells” has its general meaning in the art and refers to a subset of T cells which express CD8 on their surface, are MHC class I-restricted, and function as cytotoxic T cells. “CD8” molecules are differentiation antigens found on dendritic cells, on thymocytes and on cytotoxic and suppressor T-lymphocytes. CD8 antigens are members of the immunoglobulin supergene family and are associative recognition elements in major histocompatibility complex class I-restricted interactions.

As used herein the term “memory CD8+ T cells” has its general meaning in the art and refers to a subset of T CD8+ cells that have previously encountered and responded to their cognate antigen. Upon a second encounter with their cognate antigen, memory CD8+ T cells mount a faster and stronger immune response than the one associated with a first time antigen-encounter. As used herein the term “antigen-induced memory CD8+ T cell” refers to a subset of memory T CD8+ cells that is generated following stimulation by its cognate antigen. The term “antigen-induced memory CD8+ T cell” is defined in opposition with cytokine-induced memory CD8+ T cells.

The term “CCL5” has its general meaning in the art and refers to Chemokine (C-C motif) ligand 5. CCL5 is also known as MuRantes, RANTES, Scya5, SISd, or TCP228.

As used herein the term “NKG2D” has its general meaning in the art. In mouse the term NKG2D refers to killer cell lectin-like receptor subfamily K, member 1.

As used herein, a “sample” is a sample that comprises one or more antigen-specific T lymphocytes. As such, the term “biological sample” is used interchangeably herein with “cell sample.” A “sample” encompasses a variety of sample types obtained from an individual and can be used in a subject diagnostic or monitoring assay. A biological sample encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents (e.g., heparinization of blood samples); washed; or enrichment for CD8+ T lymphocytes. The term “sample” encompasses a clinical sample, and also includes cells in culture, tissue samples, organs, and the like. The term “sample” also includes preserved samples, including cryopreserved tissues, cryopreserved cell samples, and the like.

In some embodiments, the sample is a murine sample or a human sample. As used herein, the term “murine sample” refers to a sample which comprises cells isolated from mouse and alternatively a “human sample” refers to a sample which comprises cells from human.

The terms “antigen” is well understood in the art and refer to the portion of a macromolecule which is specifically recognized by a component of the immune system, e.g., an antibody or a T-cell antigen receptor. As used herein, the term “antigen” encompasses antigenic epitopes, e.g., fragments of an antigen which are antigenic epitopes. Epitopes are recognized by antibodies in solution, e.g. free from other molecules. Epitopes are recognized by T-cell antigen receptor when the epitope is associated with a class I or class II major histocompatibility complex molecule. Typically, the term “antigen” encompasses “antigen associated with a pathogenic organism”, “tumor associated antigen” and “allergen”. “An antigen associated with a pathogenic organism,” as used herein, is a macromolecule (e.g. a polypeptide or a fragment thereof) that is normally a part of a pathogenic organism, or is produced by a pathogenic organism. An antigen associated with a pathogenic organism is in some embodiments isolated from a naturally-occurring pathogenic organism. The term “tumor-associated antigen” is a term well understood in the art, and refers to molecules that are differentially expressed in tumor cells relative to non-cancerous cells of the same cell type. As used herein, “tumor associated antigen” includes not only complete tumor-associated antigens, but also epitope-comprising portions (fragments) thereof. An “allergen” as used herein refers to a molecule capable of provoking an immune response characterized by production of IgE. Thus, in the context of this invention, the term “allergen” refers to an antigen which triggers, in an individual who is susceptible to such (e.g., an individual who has been sensitized to the antigen), an allergic response which is mediated by IgE antibody. “Allergens” include fragments of allergens and haptens that function as allergens.

Standard methods for isolating CD8+ T cells are well known in the art. For example the methods may consist in collecting the population of CD8+ T cells present in the sample by using a binding partner directed against a specific surface marker of the CD8+ T cells (e.g. a CD8 polypeptide). In a particular embodiment, the methods of the invention comprise bringing the sample into contact with a binding partner capable of selectively interacting with CD8+ T cells present in said sample. The binding partner may be an antibody that may be polyclonal or monoclonal, preferably monoclonal, directed against the specific surface marker of the CD8+ T cells. Typically said surface marker is CD8. In another embodiment, the binding partner may be an aptamer.

In some embodiments, the population of memory CD8+ T cells are isolated from the sample by using a set of binding partners capable of selectively interacting with said cells. Typically, the set of binding partners may comprise a binding partner for CD8, and at least one binding partner specific for CD44, CD45RO/RA or CXCR3.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention or a fragment thereof can be prepared and isolated using any technique that provides for the production of antibody molecules by continuous cell lines in culture. Techniques for production and isolation include but are not limited to the hybridoma technique; the human B-cell hybridoma technique; and the EBV-hybridoma technique.

In another embodiment, the binding partner may be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consist of conformationally constrained antibody variable regions displayed by a platform protein, such as E. coli Thioredoxin A, that are selected from combinatorial libraries by two hybrid methods.

The binding partners of the invention such as antibodies or aptamers, may be labelled with a detectable molecule or substance, such as a fluorescent molecule, a radioactive molecule or any others labels known in the art. Labels are known in the art that generally provide (either directly or indirectly) a signal.

As used herein, the term “labelled”, with regard to the antibody or aptamer, is intended to encompass direct labelling of the antibody or aptamer by coupling (i.e., physically linking) a detectable substance, such as a radioactive agent, a metal or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance.

An antibody or aptamer of the invention may be labelled with a radioactive molecule by any method known in the art. For example radioactive molecules include but are not limited radioactive atom for scintigraphic studies such as I123, I124, In111, Re186, Re188.

An antibody or aptamer of the invention may be labelled with a metallic chemical element such as lanthanides. Lanthanides offer several advantages over other labels in that they are stable isotopes, there are a large number of them available, up to 100 or more distinct labels, they are relatively stable, and they are highly detectable and easily resolved between detection channels when detected using mass spectrometry. Lanthanide labels also offer a wide dynamic range of detection. Lanthanides exhibit high sensitivity, are insensitive to light and time, and are therefore very flexible and robust and can be utilized in numerous different settings. Lanthanides are a series of fifteen metallic chemical elements with atomic numbers 57-71. They are also referred to as rare earth elements. Lanthanides may be detected using CyTOF technology. CyTOF is inductively coupled plasma time-of-flight mass spectrometry (ICP-MS). CyTOF instruments are capable of analyzing up to 1000 cells per second for as many parameters as there are available stable isotope tags.

Alternatively, the antibodies against the surface markers are already conjugated to a fluorophore (e.g. FITC-conjugated and/or PE-conjugated).

The aforementioned assays may involve the binding of the binding partners (ie. Antibodies or aptamers) to a solid support. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like. The solid surfaces are preferably beads. Beads may be made of different materials, including but not limited to glass, plastic, polystyrene, and acrylic. In addition, the beads are preferably fluorescently labelled. In a preferred embodiment, fluorescent beads are those contained in TruCount™ tubes, available from Becton Dickinson Biosciences, (San Jose, Calif.).

According to the invention, methods of flow cytometry are preferred methods for isolating and quantifying of CD8+ T cells in the sample. For example, fluorescence activated cell sorting (FACS) may be therefore used to separate in the supernatant the desired CD8+ T cells. In another embodiment, magnetic beads may be used to isolate CD8+ T cells (MACS). For instance, beads labelled with monoclonal specific antibodies may be used for the positive selection of CD8+ T cells. Other methods can include the isolation of CD8+ T cells by depletion the cells that are not of interest (negative selection).

Any of a variety of methods can be used to detect the expression of CCL5 or NKG2D in the isolated population of the CD8+ T cell. Typically, the expression may be detected at the protein level or at the mRNA level.

Typically, the determination comprises contacting the sample with selective reagents such as probes, primers or ligands, and thereby detecting the presence, or measuring the amount, of polypeptide or nucleic acids of interest originally. Contacting may be performed in any suitable device, such as a plate, microtiter dish, test tube, well, glass, column, and so forth In specific embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the sample.

In a particular embodiment, the expression may be detected by determining the quantity of mRNA. Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of Amplification include ligase chain reaction (LCR), transcription-mediated amplification (TMA), strand displacement amplification (SDA) and nucleic acid sequence based amplification (NASBA). Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e.g. avidin/biotin). Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate). The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

Detection at the protein level typically comprises contacting the population of CD8+ T with a binding partner capable of selectively interacting with CCL5 or NKG2D. The binding partner is generally an antibody that may be polyclonal or monoclonal (preferably a monoclonal antibody), or an aptamer as above described. Flow cytometry methods as above described may be particularly suitable, especially for detecting the expression of NKG2D.

In a particular embodiment, the 2E9 antibody as described via https://migratech.inserm-transfert.fr/srv/tech/2/0vue2.asp?n=363 may be used to detect murine CCL5 at the protein level. In another particular embodiment, clones 21445 (R&D System), 2D5 (BectonDickinson) and VL1 (eBioscience) may be used to detect human CCL5 at the protein level

In some embodiments, both the expressions of CCL5 and NKG2D are determined.

In some embodiments, the method of the invention comprises a step consisting of determining whether the population of antigen-induced memory CD8+ T cells is specific for an antigen (i.e. determining whether a subpopulation of antigen-induced memory CD8+ T cells specific for an antigen exist in the general population of antigen-induced memory CD8+ T cells isolated in the sample). Any well known method in the art may be used for determining whether a T cell is specific of antigen.

In some embodiments, when a peptide epitope of the antigen of interested is known, it can be loaded on MHC class 1 tetramers, and the isolated antigen-induced memory CD8+ T cells is bringing into contact with said tetramers. Tetramers assays are well known in the art. To produce tetramers, the carboxyl terminus of an MHC molecule, such as, for example, the HLA A2 heavy chain, is associated with a specific peptide epitope or polyepitope, and treated so as to form a tetramer complex having bound hereto a suitable reporter molecule, preferably a fluorochrome such as, for example, fluoroscein isothiocyanate (FITC), phycoerythrin, phycocyanin or allophycocyanin. The tetramers produced bind to the distinct set of CD8+ T cell receptors (TcRs) on a subset of CD8+ T cells to which the peptide is MHCI restricted. There is no requirement for in vitro T cell activation or expansion. Following binding, and washing of the T cells to remove unbound or non-specifically bound tetramer, the number of CD8+ cells binding specifically to the HLA-peptide tetramer may be quantified by standard flow cytometry methods, such as, for example, using a FACSCalibur Flow cytometer (Becton Dickinson). The tetramers can also be attached to paramagnetic particles or magnetic beads to facilitate removal of non-specifically bound reporter and cell sorting. Such particles are readily available from commercial sources (eg. Beckman Coulter, Inc., San Diego, Calif., USA). Tetramer staining does not kill the labeled cells; therefore cell integrity is maintained for further analysis.

In some embodiments, when the epitope have not been identified, the isolated antigen-induced memory CD8+ T cell may be brought into contact with the antigen of interest (or the cell infected by the virus expressing said antigen) and the activation of T CD8+ cells may be then detected. Typically, the activation may be detected by detecting a level of a secreted factor produced by an antigen-specific CD8+ T cells. A detectable factor (e.g., a molecule that is produced and/or secreted by an antigen-specific T lymphocyte in response to signalling via the T cell antigen receptor), e.g., a secreted factor, a cell-surface marker, etc., that is produced by an antigen specific CD8+ T cells includes, but is not limited to, a cell-surface molecule such as a T cell antigen receptor (TCR); and secreted factors such as IFN-gamma, IL-2, IL-4, IL-10, TNF-alpha, MIP-la, granzymes, and perforin. In some embodiments, two or more such detectable markers are detected in a single assay. In some of these embodiments, the secreted factor is IFN-gamma. In other embodiments, the antigen-specific T lymphocyte is detected by detecting a level of a cell surface marker, e.g., an antigen specific T-cell receptor (TCR). In those embodiments in which a secreted factor is detected, the method of detection will generally be an immunological assay for detecting specific antibody-antigen interactions, e.g., an enzyme-linked immunosorbent assay (ELISA), an enzyme linked immunosorbent spot (ELISPOT) assay, an intracellular staining (ICS) assay; and the like. In many embodiments, a capture agent that binds the secreted factor is immobilized onto an insoluble support. Suitable capture agents include, but are not limited to, a cytokine binding reagent, such as a capture antibody specific for the cytokine to be detected. The captured secreted factor (e.g., a cytokine such as IFN-gamma) is visualized by a detection reagent. In some embodiments, the detection reagent is a second cytokine binding reagent (e.g., a second antibody specific for the captured factor) free in solution that is conjugated to enzyme that produces a detectable product upon acting on an appropriate substrate. In other embodiments, the detection reagents is directly labeled, e.g., with a fluorochrome or other detectable moiety, with colored beads, or a ligand such as biotin that can be detected with tertiary reagent that is labeled as above (with a fluorochrome, bead or enzyme). In some embodiments, two or more different capture reagents are employed, each of which captures a different secreted factor. In these embodiments, each different secreted factor will be detected with a different detection agent, each of which produces a distinguishable detectable signal. In some embodiments, the assay is an ELISPOT assay. In these embodiments, an antibody is immobilized on an insoluble support, e.g., a well of a 96-well nitrocellulose plate. To the well is added antigen and the isolated antigen-induced memory CD8+ T cell, forming a test sample. The test sample is kept under appropriate conditions that permit synthesis of secreted factor(s) by an antigen specific CD8+ T cells. Suitable conditions are known to those skilled in the art, and are generally 37° C. in an atmosphere containing 5% C02. After a suitable time, the level of secreted factor that has been secreted by the cells and captured by the immobilized antibody is detected, as described above.

The method of the invention may be useful in a wide variety of diagnostic assays, clinical studies (including preclinical studies), and monitoring assays.

In some embodiments, the method may be is useful for determining whether a subject has developed a memory T cell response in a context of an infection with a pathogenic organism or a disease such as cancer, allergy, an autoimmune disorder, etc. Thus, e.g., the method is useful for detecting a population of antigen-induced memory CD8+ T cells that is specific for antigens associated with a pathogen; for a tumor-associated antigen; for an allergen; or for a self antigen.

In some embodiments, the subject is a human or a mouse.

Polypeptides and peptide epitopes associated with microbial pathogens are known in the art and include, but are not limited to, antigens associated with human immunodeficiency virus (HIV), e.g., HIV gp120, or an antigenic fragment thereof; cytomegalovirus antigens; Mycobacterium antigens (e.g., Mycobacterium avium, Mycobacterium tuberculosis, and the like); Pneumocystic carinii (PCP) antigens; malarial antigens, including, but not limited to, antigens associated with Plasmodium falciparum or any other malarial species, such as 41-3, AMA-1, CSP, PFEMP1, GBP-130, MSP-1, PFS-16, SERP, etc.; fungal antigens; yeast antigens (e.g., an antigen of a Candida spp.); toxoplasma antigens, including, but not limited to, antigens associated with Toxoplasma gondii, Toxoplasma encephalitis, or any other Toxoplasma species; Epstein-Barr virus (EBV) antigens; and the like. Additional antigens of interest include antigens to which a subject may have been exposed, including, but not limited to, Mycobacterium bovis (Bacille Calmette-Guerin); poxvirus antigens; and the like.

Allergens of interest according to the present invention include antigens found in foods such as fruits (e.g., melons, strawberries, pineapple and other tropical fruits), peanuts, peanut oil, other nuts, milk proteins, egg whites, shellfish, tomatoes, etc.; airborne antigens such as grass pollens, animal danders, house mite feces, etc.; drug antigens such as penicillins and related antibiotics, sulfa drugs, barbiturates, anticonvulsants, insulin preparations (particularly from animal sources of insulin), local anesthetics (e.g., Novocain), and iodine (found in many X-ray contrast dyes); insect venoms and agents responsible for allergic dermatitis caused by blood sucking arthropods such as Diptera, including mosquitos (Anopheles sp., Aedes sp., Culiseta sp., Culex sp.), flies {Phlebotomus sp., Culicoides sp.) particularly black flies, deer flies and biting midges, ticks {Dermmacenter sp., Omithodoros sp., Otobius sp.), fleas (e.g., the order Siphonaptera, including the genera Xenopsylla, Pulex and Ctenocephalides felis felis); and latex. The specific allergen may be any type of chemical compound such as, for example, a polysaccharide, a fatty acid moiety, a protein, etc.

Tumor-associated antigens (or epitope-containing fragments thereof) include, but are not limited to, MELOE, MAGE-2, MAGE-3, MUC-1, MUC-2, HER-2, high molecular weight melanoma-associated antigen MAA, GD2, carcinoembryonic antigen (CEA), TAG-72, ovarian-associated antigens OV-TL3 and MOV1 8, TUAN, alpha-feto protein (AFP), OFP, CA-125, CA-50, CA-19-9, renal tumor-associated antigen G250, EGP-40 (also known as EpCAM), SI00 (malignant melanoma-associated antigen), p53, and p21ras. In some embodiments, a fragment of a TAA is used. A 5 synthetic analog of any TAA (or epitope thereof), including any of the foregoing, may be used. Furthermore, combinations of one or more TAAs (or epitopes thereof) may be used.

Autoantigens (“self antigens”) include but are not limited to, myelin basic protein or a fragment of myelin basic protein; proteolipid protein (PLP); HSP70; Ku (p70/p80) autoantigen, or its 80-kd subunit protein; the nuclear autoantigens La (SS-B) and Ro (SS-A); scleroderma antigens Rpp 30, Rpp 38 or Sc1-70; the centrosome autoantigen PCM-1; polymyositis-scleroderma autoantigen; scleroderma (and other systemic autoimmune disease) autoantigen CENP-A; U5, a small nuclear ribonucleoprotein (snRNP); the 100-kd protein of PM-Scl autoantigen; the nucleolar U3- and Th(7-2) ribonucleoproteins; the ribosomal protein L7; hPopl; and a 36-kd protein from nuclear matrix antigen; and the like.

In some embodiments, a subject method is useful for identifying to which allergen(s) an individual will be sensitive, e.g., to which allergen(s) an individual exhibits an allergic reaction. Thus, e.g. T lymphocytes in a biological sample from an individual are exposed to a panel of allergens, so that the allergen(s) to which T lymphocytes in the sample are reactive can be identified.

In other embodiments, the methods are useful for determining the efficacy of a treatment for a disorder. In some embodiments, the present invention provides methods of determining the response of an individual to treatment for an infection with a pathogen. In other embodiments, the present invention provides methods of determining the response of an individual to treatment for cancer. In other embodiments, the present invention provides methods of determining the response of an individual to treatment for allergy.

In other embodiments, the methods are useful for clinical studies, e.g., to compare the level of antigen-induced memory CD8+ T cells from one individual to another, e.g., in response to treatment, in the absence of treatment, etc.

In other embodiments, a subject method is useful for testing efficacy of a vaccine (e.g. for the prophylactic treatment of an infection or for the treatment of cancer). For example, the level of antigen-induced memory CD8+ T cells specific for an antigen of a vaccine preparation, in a sample from a subject who has received the vaccine is compared to the level of antigen-induced memory CD8+ T cells specific for the same antigen in a biological sample from a subject individual who has received a placebo, when the level determined in the subject administered with the vaccine preparation is higher than the level determined with the subject administered with the placebo indicates that the vaccine preparation was efficient. Alternatively, the level of antigen-induced memory CD8+ T cells specific for an antigen included in a vaccine preparation, is determined after the administration of the vaccine preparation to the subject by comparing the level of CCL5 or NKG2D containing, antigen-induced memory CD8+ T cells before and after the administration to the subject, an increase in the level of the CCL5 or NKG2D containing, antigen-induced memory CD8+ T cells indicates that the vaccine preparation was efficient. In another example (e.g. for vaccine preparation based on attenuated viruses), the level of the whole population of antigen-induced memory CD8+ T cells is determined after the administration of the vaccine preparation to the subject and then compared to the level of the whole population of antigen-induced memory CD8+ T cells determined before the administration to the subject, wherein an increase in the level of the whole population of antigen-induced memory CD8+ T cells indicates that the vaccine preparation was efficient. The method may be thus particularly suitable for screening combinations of antigens and/or immunoadjuvants for the preparation of vaccine preparation.

The present invention also relates to kits suitable for performing the method of the invention comprising means for isolating CD8+ T cell in a sample and means for detecting the expression of CCL5 or NKG2D. In a particular embodiment, the kit comprises an antibody as above described (e.g. 2E9 antibody as described via https://migratech.inserm-transfert.fr/srv/tech/2/0vue2.asp?n=363).

The invention will be further illustrated by the following figures and examples. However, these examples and figures should not be interpreted in any way as limiting the scope of the present invention.

### FIGURES

FIG. 1: Generation of different CD8⁺ memory subsets. (A) Strategies to generate different memory subsets. Non-immunized F5 mice contain mainly naive cells. NP-68 immunized F5 mice contain T_{IM}. Pathogen-induced F5 memory cells were obtained by transferring sorted naive F5 CD8⁺ T cells into C57Bl/6 mice followed by immunization with either influenza virus bearing the NP68 peptide (Flu-NP68) or vaccinia virus bearing the NP68 peptide (VV-NP68). (B) Analysis of the expression of memory markers (CXCR3 and CD44) among CD8⁺ T cells 40 days after immunization. Only F5 CD8⁺ T cells are depicted.

FIG. 2: Expression of CCL5 by CD8⁺ memory subsets. Intracellular staining of CCL5 in naive cells, T_{IM} and pathogen-induced memory cells. Only F5 CD8⁺ T cells are depicted. This side by side comparison of subsets is representative of multiple separate phenotyping experiments. Filled histogram: control naive CD8⁺ T cells, Red histogram: memory cells.

FIG. 3: Characterization of HP memory-phenotype CD8⁺ T cells. (A) Strategy to generate HP memory CD8⁺ T cells. Transferred cells (CD45.1⁻, CD45.2) could be differentiated from host CD8 (CD45.1⁺, CD45.2) by CD45.1 and CD45.2 double staining (B, C) Analysis of memory phenotype (CD44, CXCR3) and proliferation (visualized by CTV signal dilution), of transferred CD8⁺ T cells. The origin of donor cells and hosts are indicated at the top of each column. D: Divided, ND: Not Divided, . . . : positivity threshold. Analysis was performed 12 days (B) or 32 days (C) after transfer. One representative experiment out of two is shown.

FIG. 4: HP memory-phenotype CD8⁺ T cells do not express CCL5 protein. CCL5 expression among transferred CD8⁺ T cells CD45.1⁻ CD45.2⁺ 12 days (A) or 32 days (B) after transfer. The origin of donor cells and hosts are indicated. Histograms show the mean fluorescence intensity of CCL5. Filled histogram: host's cell population expressing CCL5, open histogram: transferred CD8⁺ T cells. D: Divided, ND: Not Divided. One representative experiment out of two is shown.

FIG. 5: Intracellular CCL5 expression by memory CD8⁺ subsets. (A) The percentage of CD8⁺ T cells expressing detectable intracellular CCL5 among memory CD8⁺ subsets is represented. (B) Table summarizing the expression of CCL5 by memory CD8⁺ subsets. −: unexpressed, +/−: expressed by a fraction of cells, +: expressed by all cells.

FIG. 6: CCL5 mRNA levels among memory CD8⁺ subsets. (A) Populations of interest were sorted using a FACSAria cytometer. F5 naive CD8⁺ T cells are CD8⁺ CD44^{lo} from unimmunized F5 mice. TIM are CD8⁺ CD44^{int} from NP68-immunized F5 mice. HP memory cells derived from transferred naive C57Bl/6 CD8⁺ T cells into lymphopenic C57Bl/6 mice were sorted after 32 days as CTV^{lo} CD8⁺ cells. Pathogen-induced F5 memory cells are F5 cells transferred into C57Bl/6 mice immunized with Flu-NP or VV-NP and sorted at day 40 (CD45.1⁻, CD45.2⁺, CD8⁺, and CD44^{hi}). Total mRNAs were extracted and quantitative PCR was performed on cDNAs after reverse transcription. For each memory CD8+ subset, CCL5 mRNA levels presented are relative to ubiquitin mRNA level. (B) Table summarizing the CCL5 mRNA levels among memory CD8⁺ subsets. −: low level, ++ and +++: high level.

FIG. 7: CCL5 protein expression among memory phenotype CD8⁺ T cells. (A) Memory phenotype cells from unimmunized or immunized C57Bl/6 mice were analyzed for their expression of CCL5 protein. (B) The number of cells expressing CCL5 protein was assessed in memory phenotype CD8⁺ T cells from six naive (open histogram) or twelve immunized (black histogram) C57Bl/6 mice.

FIG. 8: CCL5 and NKG2D are co-expressed by mouse memory CD8 T cells. (A) At different time point post VV-NP infection (2×10⁵ PFU), CD8 T cells from the blood were assessed for their expression of CCL5 and NKG2D. (B) Linear regression between the number of CCL5+ and NKG2D+ memory phenotype cells recovered in the blood of individual VV-infected mice at different time points post-infection. Result for 10 mice are shown. Correlation index (r²) is shown for each graph.

### EXAMPLE 1

Material & Methods

Mice and Generation of CD8⁺ Memory Populations

C57Bl/6 mice were purchased from Charles Rivers. F5 TCR-transgenic mice were gifts from D. Kioussis (National Institute for Medical Research, London, U.K)¹⁸. The F5 TCR recognizes the influenza virus-derived NP68 peptide in the context of H-2D^{b}. Mice were bred in our animal facility, “Plateau de Biologie Expérimentale de la Souris” (PBES-AniRA, UMS3444/US8, SFR BioSciences Gerland Lyon Sud).

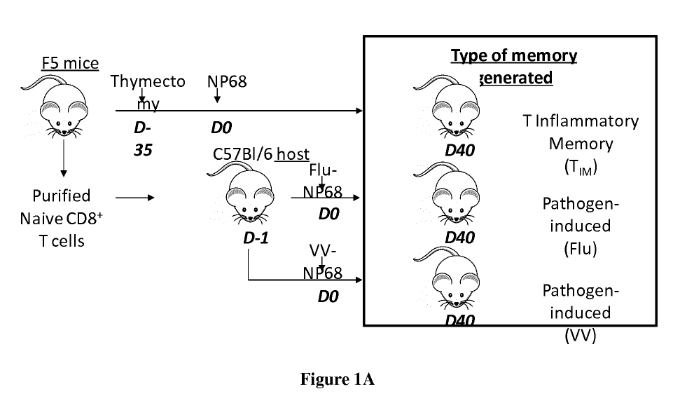

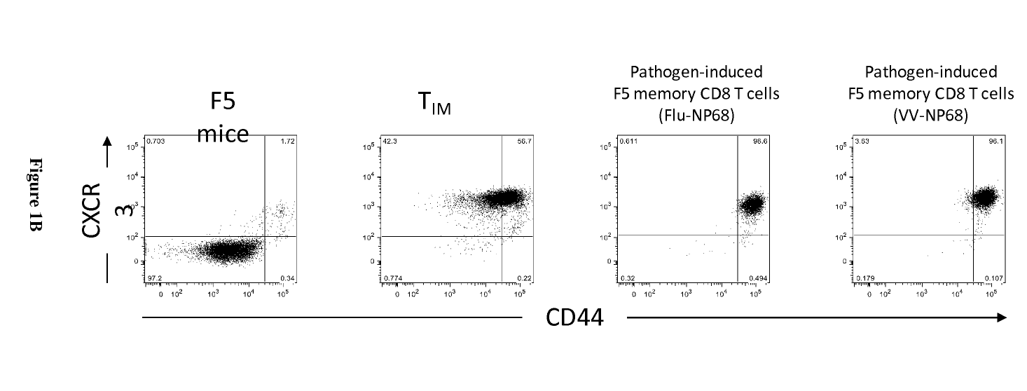

To generate CD8⁺ T inflammatory memory cells (T_{IM}), 10 weeks old thymectomized F5 mice were immunized twice I.P (intraperitonealy) with NP68 peptide (50 nmol in PBS), as previously described¹⁹. T_{IM} were generated by J. Mafille (FIG. 1A).

To generate pathogen-induced F5 memory CD8⁺ T cells, 2×10⁵ F5 naive CD8⁺ T cells were transferred I.V. (intravenously) into C57Bl/6 mice. The next day, animals were immunized I.N. (intranasaly) either with Influenza-NP68 (2×10⁵ TCID 50) or Vaccinia-NP68 (2×10⁵ PFU) viruses. Pathogen-induced F5 memory CD8⁺ T cells were generated either by S. Djebali or myself.

To generate HP memory CD8⁺ T cells, naive donor cells (CD45.1⁻, CD45.2⁺) from either C57Bl/6 or F5 mice were transferred I.V. into C57Bl/6 mice (CD45.1⁺, CD45.2⁺) rendered lymphopenic the day before the transfer by a sub-lethal irradiation (600 rad). As a control, non irradiated hosts were transferred with naive donor cells. Naive donor cells to be transferred were obtained by disaggregation of spleen from donor mice through a 100 μm Cell strainer (BD Falcon). CD8⁺ T cells were purified by negative selection using the Automacs Technology according to the manufacturer instructions (Miltenyi). Cells were then labeled with the Invitrogen CellTrace Violet kit (Invitrogen Molecular Probes, cat No C34557): cells were resuspended at a concentration of 5×10⁶ cells/ml in DMEM complete medium containing 5 μM CellTrace freshly dissolved and incubated 20 minutes at 37° C., protected from light. After one wash, cells were stained with specific antibodies for CD8 and CD44. Naive CD8⁺ T cells CTV-labeled (CTV⁺, CD8⁺, CD44^{lo}) were sorted using a FACSAria cytometer (BD Biosciences) and resuspended at 1×10⁷ cells/ml in PBS. 2×10⁶ cells were injected I.V. in anesthetized recipient mice. Transferred mice were given antibiotics (Bactrim) to prevent infections.

Flow Cytometry

Splenocytes were isolated by disaggregation of spleens through 100 μm cell strainer (BD Falcon) and washed once in Facs stain (PBS, 1% FCS, 0.09% NaN₃⁺). For staining of surface markers, splenocytes were stained with fluorochrome-conjugated antibodies diluted in Facs stain. After 45 min of incubation at 4° C. protected from light, cells were washed and resuspended in Facs stain. To detect intracellular CCL5, cells were first stained as indicated above and then fixed and permeabilized (cytofix/cytoperm, BD Biosciences) before staining for one hour at room temperature protected from light with a aCCL5 antibody (1 μg/ml) diluted in permwash buffer (BD Biosciences). Cells were then washed and resuspended in permwash buffer. Acquisitions were made using a LSRII cytometer (BD Biosciences) and analyses were done using FlowJo software (Tristar). Antibodies used are αCD8 APC Alexa 780 (clone 53-6.7), αCXCR3 APC (CXCR3 173) from eBiosciences, αCD44 PE (IM 7.8.1), αCD45.1 FITC (A20), αCD45.2 PerCP Cy5.5 (104) from BD Biosciences and aCCL5 APC A647 (home made).

Quantitative PCR

CD8⁺ T cells were sorted using a FACSAria, based on the following markers: naive cells (CD8⁺, CD44^{lo}), T_{IM} (CD8⁺, CD44^{int}), HP memory cells (CD8⁺, CD45.2⁺ and CTV⁻), pathogen-induced F5 memory cells (CD8⁺, CD44⁺ and CD45.2⁺). Total mRNAs were extracted using the RNeasy kit according to the manufacturer instructions (QIAGEN). Total mRNAs were reverse transcribed in cDNAs using the iScript™ cDNA synthesis kit according to the manufacturer instructions (Biorad). Quantitative PCR was performed on total cDNAs by using CCL5 primers and an ABI Prism 7700 (Perkin Elmer). Relative levels of the target sequence were calculated using the AA cycle threshold method.

Results

Generation of Different Memory CD8⁺ Subsets Using the F5 Model.

The memory compartment is composed of heterogeneous memory CD8⁺ T cell subsets, reflecting the diversity of conditions leading to memory generation. Since it is difficult to analyse antigen specific naive and memory T cells due to their low frequency, we took advantage of the F5 model. F5 mice bear a transgenic TCR, meaning that all the CD8⁺ T cells from F5 mice bear the same TCR, with the same antigenic specificity. This TCR recognizes the peptide derived from influenza nucleoprotein (NP68) loaded onto H-2D^{b} MHC class I molecule. The CD8⁺ T cell population of non-immunized F5 mice is mainly constituted of naive cells that do not express the memory markers CD44 and CXCR3 (FIG. 1).

Using the F5 model, we are able to differentiate naive F5 CD8⁺ T cells into different memory CD8⁺ subsets. Immunization of thymectomized F5 mice twice with NP68 peptide leads to the generation of T inflammatory memory cells (T_{IM}) (FIG. 1A). T_{IM} express high levels of CXCR3 and intermediate levels of CD44 (FIG. 1).

To generate pathogen-induced F5 memory CD8⁺ T cells, naive CD8⁺ T cells from F5 mice were sorted using a cell-sorter and transferred into C57Bl/6 mice. The next day, hosts were immunized I.N. with either influenza (Flu-NP68) or vaccinia (VV-NP68) viruses, both bearing the NP68 peptide (FIG. 1A). 40 days later memory cells were studied. Pathogen-induced F5 memory CD8⁺ T cells express high levels of CXCR3 and CD44. Memory cells generated in response to these two viruses are very similar in terms of phenotype (FIG. 1B) or functions.

CCL5 is Expressed by Antigen-Induced Memory CD8⁺ T Cells Subsets.

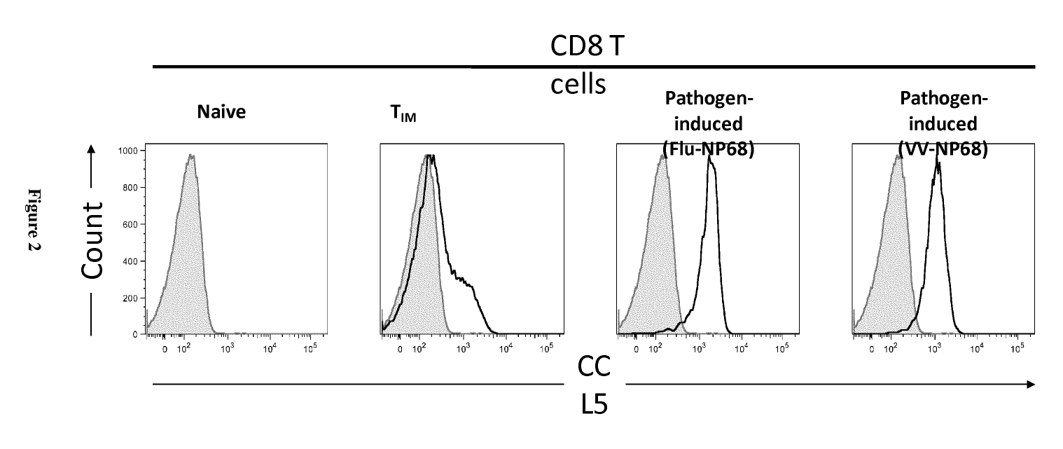

To test if CCL5 expression discriminates between memory CD8⁺ subsets, we analysed CCL5 protein expression among different memory T cell subsets in parallel. Splenocytes were fixed, permeabilized and intracellular CCL5 was detected using home-made aCCL5 antibody. In naive F5 mice, the population of naive CD8⁺ T cells, characterized by low expression of CD44, does not express CCL5 protein (FIG. 2). Among T_{IM}, only a fraction of cells express CCL5 (FIG. 2). In contrast, the vast majority of pathogen-induced F5 memory CD8⁺ T cells, generated either with influenza or vaccinia viruses, express CCL5 protein (FIG. 2). Therefore, we conclude that among the antigen-induced memory CD8⁺ subsets generated using the F5 model, CCL5 is expressed by all pathogen-induced memory CD8⁺ T cells whereas it is only expressed by a fraction of T_{IM}.

C57Bl/6 Naive CD8⁺ T Cells Acquire a Memory Phenotype Through Homeostatic Proliferation.

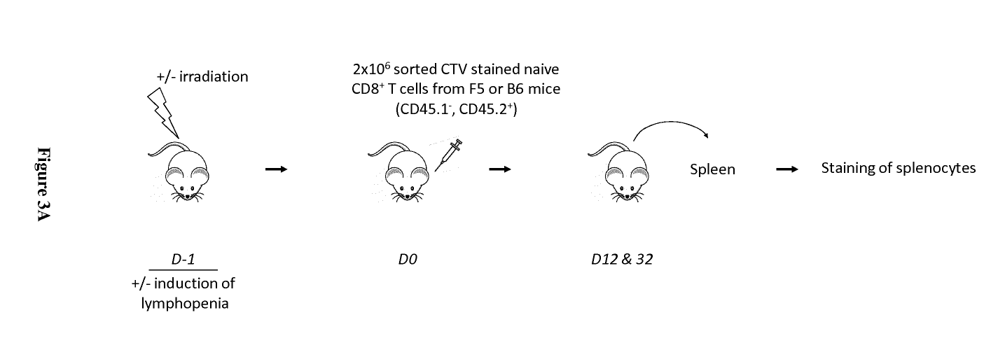

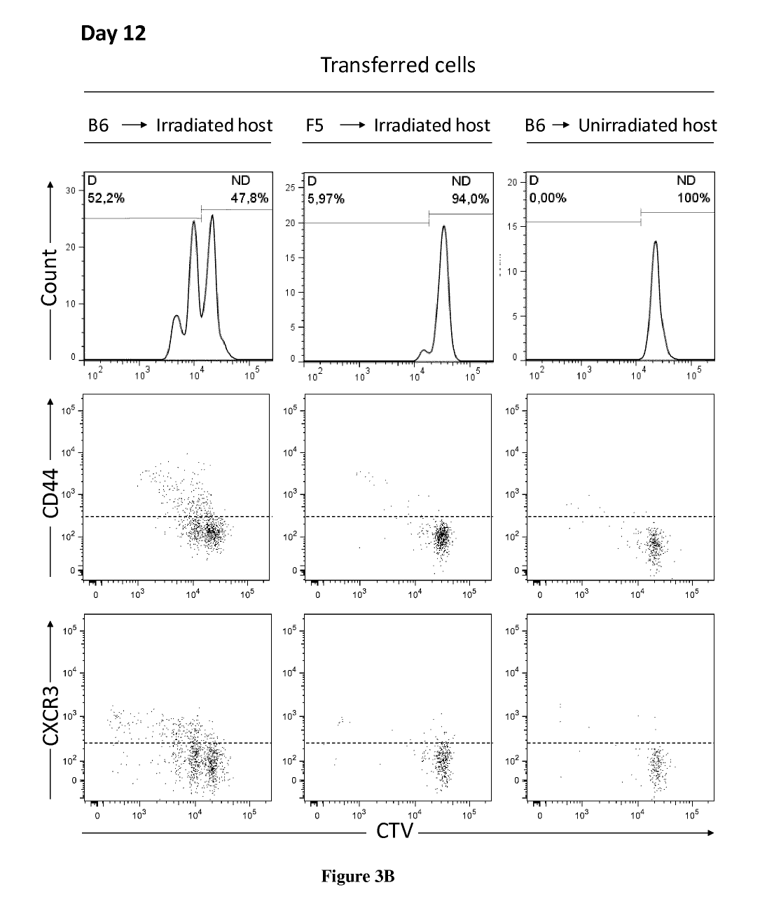

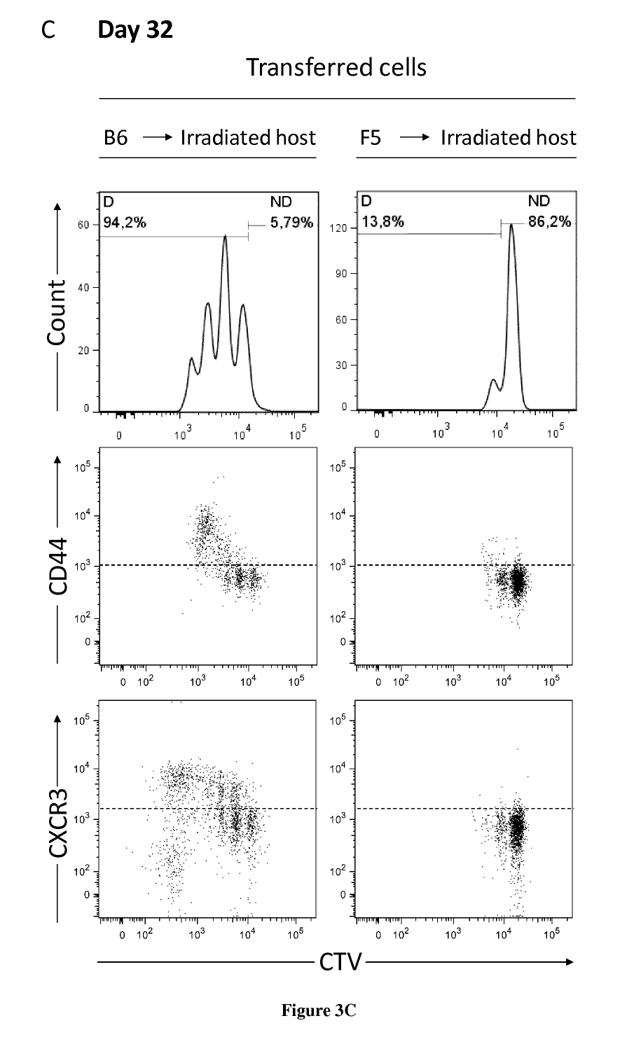

We next decided to analyse CCL5 expression by memory-phenotype cells generated through homeostatic proliferation (HP memory cells). The transfer of naive polyclonal CD8⁺ T cells from C57Bl/6 mice into C57Bl/6 host rendered lymphopenic through irradiation induces the generation of HP memory-phenotype cells⁹. To set up the experimental system, we have transferred naive CD8⁺ T cells from either C57Bl/6 or F5 mice into lymphopenic C57Bl/6 hosts. The generation of HP memory-phenotype cells is mainly dependent on cytokines (interleukin 7) but also involves a weak TCR engagement similarly to the positive selection in the thymus⁸⁻¹⁰. As we were not sure that the F5 CD8⁺ T cells would perform homeostatic proliferation in lymphopenic conditions, we also used as donor cells a polyclonal naive CD8⁺ T cell population from C57Bl/6 mice. To analyse their proliferation, naive CD8⁺ T cells were labeled with CTV before transfer (FIG. 3A). As expected, 12 days after transfer, a large proportion of transferred C57Bl/6 naive CD8⁺ T cells had proliferated, as revealed by CTV signal dilution, when transferred into lymphopenic hosts (FIG. 3B). Among proliferating cells, the expression of memory markers CD44 and CXCR3 is proportional to the number of cell divisions, indicating that among proliferating CD8⁺ T cells, some had acquired a memory phenotype (FIG. 3B). As a control, C57Bl/6 naive CD8⁺ T cells when transferred into a non-lymphopenic host (FIG. 3B) did not proliferate and do not express the memory markers CD44 and CXCR3. Only a very small fraction of transferred F5 naive CD8⁺ T cells performed one division 12 days after transfer. Therefore the level of CD44 and CXCR3 expression could not be monitored accurately (FIG. 3B). 32 days after transfer, we can observe that almost all surviving transferred C57Bl/6 naive CD8⁺ T cells have proliferated and a fraction have acquired the memory markers CD44 and CXCR3 (FIG. 3C). On the other hand, only a minority of transferred F5 naive CD8⁺ T cells had proliferated 32 days after transfer and they mainly retained their naive phenotype. Therefore, as previously demonstrated by other, we observed that homeostatic proliferation of C57Bl/6 naive CD8⁺ T cells is associated with the acquisition of CD44 and CXCR3, i.e. they acquired a memory-phenotype.

HP Memory-Phenotype CD8⁺ T Cells do not Express CCL5 Protein.

We next analysed CCL5 protein expression by HP memory CD8⁺ T cells. As expected, CCL5 is not expressed by CD8⁺ T cells that have not divided and that have retained a naive phenotype (FIG. 4). This is true for C57Bl/6 CD8⁺ T cells transferred into un-irradiated non-lymphopenic hosts and for F5 CD8⁺ T cells transferred into lymphopenic hosts (day 12 and 32). Interestingly, C57Bl/6 CD8⁺ T cells transferred into irradiated hosts and that have proliferated (day 12 and 32) did not express CCL5 at the protein level although a fraction of them had acquired a memory phenotype (FIG. 3B). As a staining positive control, CCL5 is detected in a host cell population that could correspond to NK cells, as these cells have been shown to constitutively express CCL5. Therefore, these observations indicate that unlike antigen-induced memory CD8⁺ T cells, HP memory-phenotype CD8⁺ T cells do not express CCL5 protein.

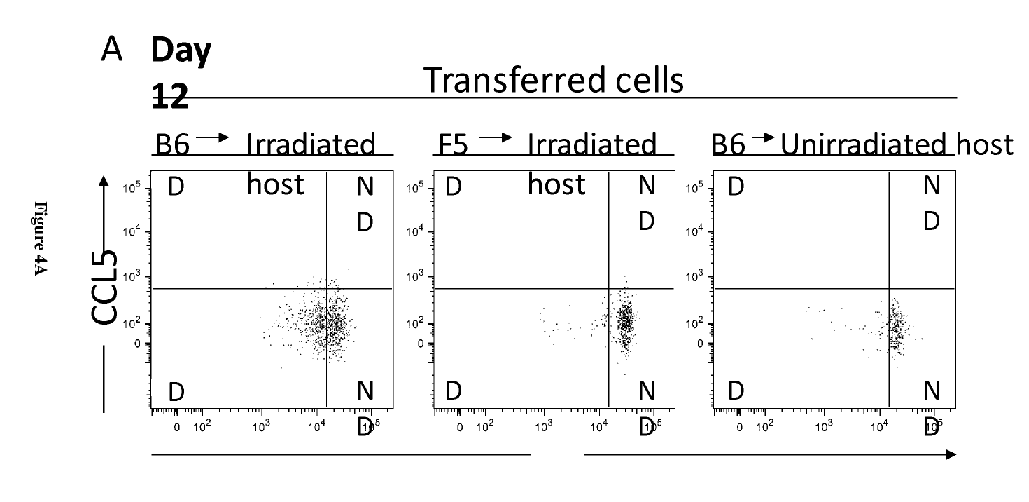

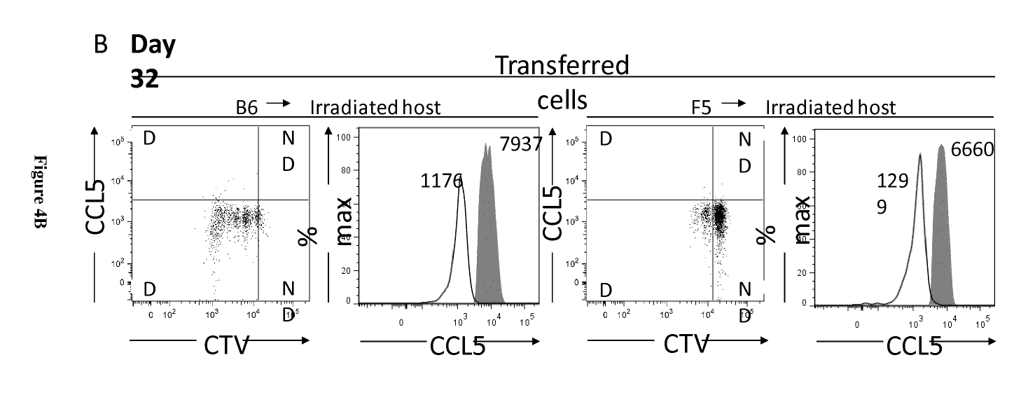

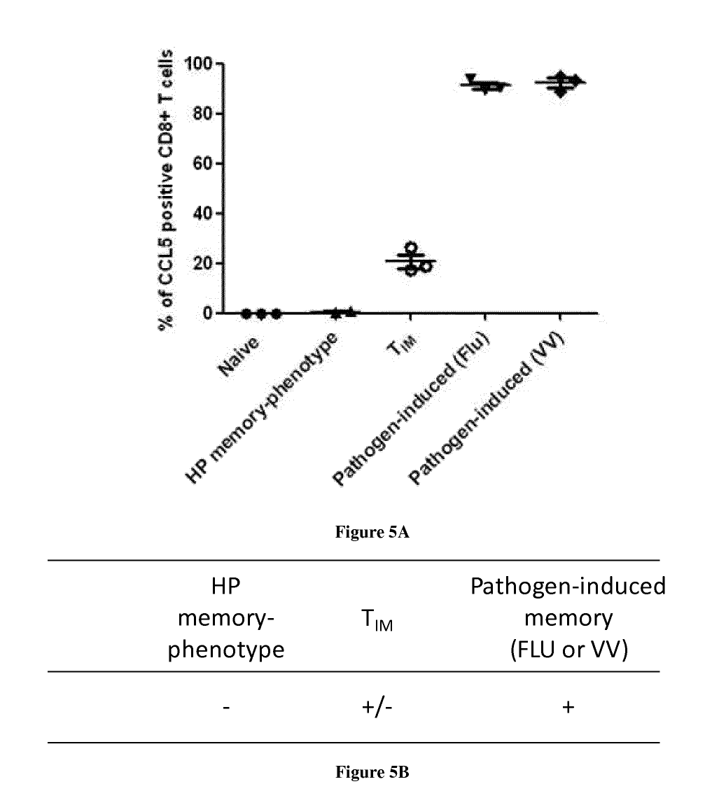

To summarize, our results indicate that among memory-phenotype CD8⁺ T cells, only bona fide antigen-induced memory CD8⁺ T cells express CCL5 protein, with all fully differentiated pathogen-induced memory cells expressing CCL5 and only a fraction (25%) of partially differentiated T_{IM}, expressing detectable CCL5 protein. In contrast, HP memory-phenotype CD8⁺ T cells, like naive CD8⁺ T cells, do not express CCL5 (FIG. 5).

CCL5 mRNA Levels Discriminate Memory CD8⁺ Subsets.

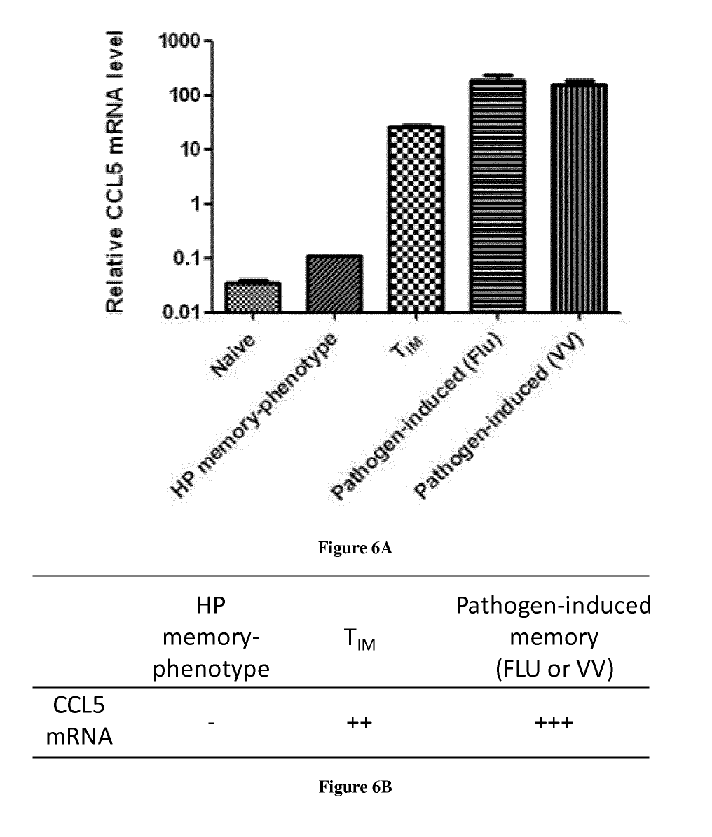

As CCL5 can be stored in memory cells as untranslated mRNA^{20, 21, 22}, we have wondered whether untranslated CCL5 mRNA was accumulating in HP memory-phenotype CD8⁺ cells. We have isolated different subsets of CD8⁺ cells (F5 naive cells, T_{IM}, HP C57Bl/6 memory-phenotype cells, pathogen-induced F5 memory cells) by cell sorting. Total mRNAs were extracted, reverse transcribed into cDNA and relative CCL5 mRNA levels were quantified by quantitative PCR and compared to ubiquitin mRNA levels (FIG. 6A). As expected, F5 naive CD8⁺ T cells express very low levels of CCL5 mRNA. Importantly, C57Bl/6 HP memory-phenotype cells also express very low levels of CCL5 mRNA (FIG. 6A). In contrast, T_{IM} and pathogen-induced F5 memory CD8⁺ T cells express at least a 100 fold increase in CCL5 mRNA levels compared with naive or HP memory-phenotype subsets (FIG. 6A). Thus homeostatic proliferation does not induce the accumulation of untranslated CCL5 mRNA stores as observed in antigen-induced memory cells.

Antigen-Induced Memory CD8⁺ T Cells can be Detected In Vivo Using CCL5 Staining

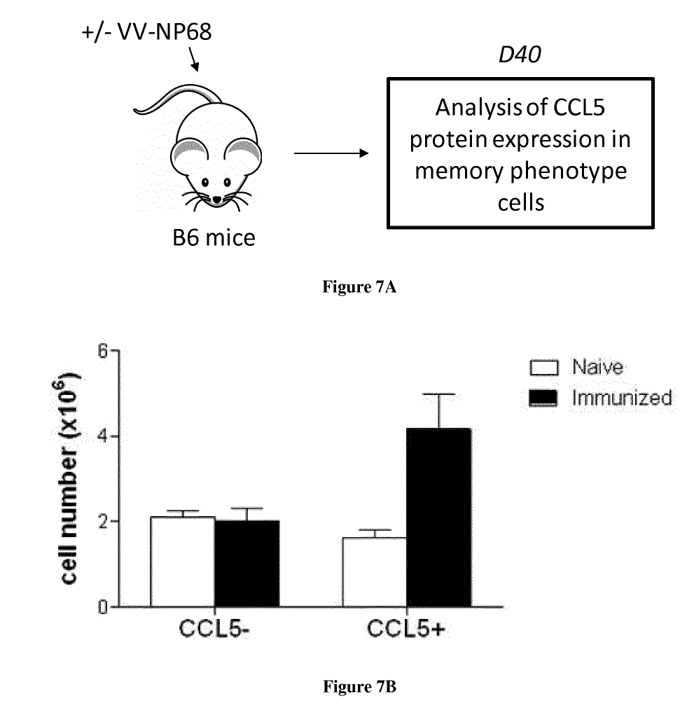

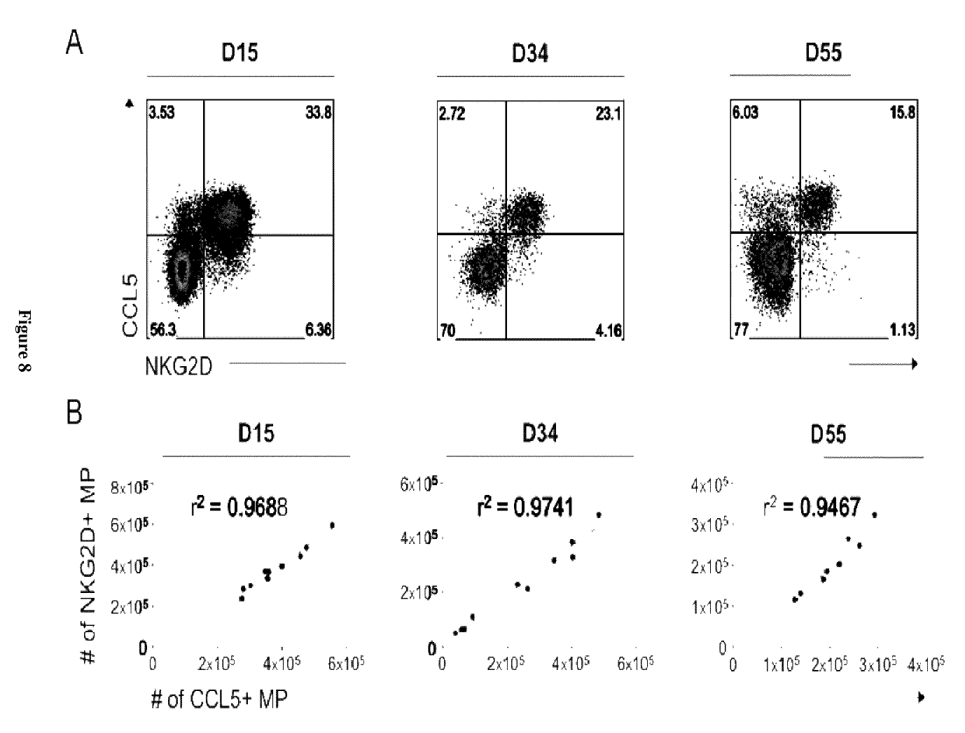

Memory-phenotype CD8⁺ T cells are found in unimmunized inbred mice housed under specific-pathogens free (SPF) conditions^{8, 23}. However, how these memory phenotype cells are generated is still poorly understood. In the pool of CD8⁺ T cells having a memory phenotype (CD44⁺CXCR3⁺) from unimmunized C57Bl/6 mice only a fraction express CCL5 protein (FIG. 7). Our results suggest that the one expressing CCL5 are antigen-induced memory CD8⁺ T cells whereas the one that do not express CCL5 are cytokine-induced memory CD8⁺ T cells. In that case, deliberate immunization with a pathogen should only induce CCL5 expressing memory cells. To test this, we immunized C57Bl/6 mice and analysed CCL5 expression by the memory phenotype (CD44^{h1}) CD8⁺ T cells (FIG. 7A). In C57Bl/6 mice immunized with VV-NP68, we observed that the number of memory (CD44⁺) CD8⁺ T cells expressing CCL5 is specifically increased, whereas the number of CCL5 negative memory phenotype cells is unchanged (FIG. 7B). This reflects the fact that these cells expressing CCL5 are antigen-induced memory CD8⁺ T cells. Thus, CCL5 protein expression allows monitoring the generation of antigen-induced memory CD8⁺ T cells in response to an infection. Finally, we identified the co-stimulatory receptor NKG2D as a new biomarker that is expressed by antigen-induced memory CD8 T cells generated in response to pathogens such as vaccinia virus in C57Bl/6 mice (FIG. 8).

Discussion

The memory compartment is composed of antigen-induced memory cells (pathogen-induced memory CD8⁺ T cells and T_{IM}) that are true memory cells and cytokine-induced memory phenotype cells (innate and HP memory phenotype CD8⁺ T cells) for which the antigen specificity is unknown. However, the phenotypic markers commonly used to characterize a memory phenotype CD8⁺ T cell (CD44, CXCR3, IFNγ) do not discriminate between antigen-induced and cytokine-induced memory CD8⁺ T cells. In this study, we demonstrated that CCL5 protein is expressed by antigen-induced but not by cytokine-induced memory CD8⁺ T cells. At the mRNA level, we demonstrated that untranslated CCL5 mRNA stores exist in antigen-induced memory CD8⁺ T cells whereas very low levels of CCL5 mRNA were detected in cytokine-induced memory CD8⁺ T cells. Therefore, our results indicate that CCL5 can be used as a biomarker to track antigen-induced memory CD8⁺ T cells generated after infection. In support, results obtained in the team show that innate memory CD8⁺ T cells, another cytokine-induced population, is devoid of CCL5 protein expression (Ventre E., 2011, under revision).

The results presented in this study were obtained using the F5 TCR transgenic mouse model to generate large numbers of memory CD8⁺ T cells. Importantly, in wild-type mice immunized with vaccinia-NP68 virus an endogenous CD8 response against vaccinia virus occurs and memory CD8⁺ T cells directed against the dominant vaccinia epitope B8R can be detected using dextramer (a complex of B8R peptide with MHC class I molecules coupled to fluorochrome that will stain CD8⁺ T cells harboring a TCR directed against the B8R peptide). Vaccinia-induced B8R-specific endogenous memory CD8⁺ T cells also express CCL5 protein (data not shown). These results indicate that the expression of CCL5 protein by pathogen-induced memory CD8⁺ T cells is also observed in non transgenic CD8⁺ T cells.

The results presented in this study with pathogen-induced memory CD8⁺ T cells were obtained using viruses (influenza or vaccinia). Therefore, it will be essential to extend these findings to other type of pathogens such as bacteria. Experiments are in progress in the laboratory to set up the use of the bacteria Listeria monocytogenes bearing NP epitope as a pathogen to generate memory CD8⁺ T cells.

The route of viral entry could have an impact on the CD8⁺ T cell response and could therefore impact the expression of CCL5 by the memory CD8⁺ T cells generated. Although we used I.N. immunization to generate pathogen-induced CD8⁺ memory T cells, previous experiments in the laboratory show that I.P. immunization also leads to the generation of pathogen-induced CD8⁺ memory T cells that express CCL5 protein, suggesting that CCL5 expression seems not dependent on viral entry site.

Memory-phenotype CD8⁺ T cells are found in unimmunized inbred mice housed under specific-pathogens free (SPF) conditions and to a lesser extend in unimmunized mice housed under germ free conditions. This suggests that in SPF mice a part of memory-phenotype CD8⁺ T cells could be generated following the encounter of environmental antigens whereas another part could be generated through cytokine-induced homeostatic proliferation^{8 23}. The analysis of CCL5 protein expression among these memory phenotype cells from unimmunized C57Bl/6 mice confirms this hypothesis. Indeed, we demonstrated that a fraction of these memory phenotype cells express CCL5 protein suggesting that these cells are antigen-induced memory cells (FIG. 7B). On the other hand, some of these memory phenotype cells do not express CCL5 protein and are therefore likely to be cytokine-induced memory cells (FIG. 7B).

Antigen-induced memory cells are generated after a strong avidity TCR stimulation of naive CD8⁺ T cells. In contrast, cytokine-induced memory CD8⁺ T cells are generated after weak avidity TCR stimulation. Indeed, HP memory-phenotype CD8⁺ T cell generation depends of IL-7 but also of TCR stimulations with self antigens. However, the TCR avidity for self antigen is very low since CD8⁺ T cells hyper responsive to self antigens are eliminated during thymic positive selection to prevent auto immune diseases. Moreover, naive CD8⁺ T cells are allowed to survive through weak TCR stimulations with self antigens without being activated⁸. Thus, our results suggest that a strong TCR stimulation is required for the acquisition of CCL5 stores.

Among antigen-induced memory cells, all the pathogen-induced CD8⁺ T cells translate CCL5 mRNA into protein whereas only a pool of cells among the T_{IM} subset expresses CCL5 protein. The T_{IM} population, as a whole, contains store of unstranslated CCL5 mRNAs, as the blockage of transcription leads to the secretion of same amounts of CCL5 protein between T_{IM} and pathogen-induced CD8⁺ T cells²². However, we do not know whether all T_{IM} cells have untranslated CCL5 mRNA stores. Therefore, it could be of interest to study CCL5 mRNA expression at the cell level among the T_{IM} subset by in situ hybridization or by single cell PCR. The less differentiate state of T_{IM} (CD44^{int}) suggests that they did not received the same signaling as pathogen-induced memory CD8⁺ T cells, especially in terms of co-stimulatory signals. This is corroborated by the fact that T_{IM} further differentiate upon antigenic re-stimulation by PAMP-matured DC⁶ or viral infection (Jubin V., in preparation). Therefore, the fact that not all the T_{IM} translate CCL5 mRNA is maybe closely related to their less differentiated state compared to pathogen-induced memory CD8⁺ T cells. Interestingly, it was demonstrated in the laboratory that among fully differentiated T_{IM} following vaccinia infection, all express CCL5 protein. This observation fits well with our hypothesis that the acquisition of CCL5 protein expression by antigen-induced memory CD8⁺ T cells depends of the strength of the signaling received during the primary immune response.

Memory CD8⁺ T cells produce large amounts of IFNγ upon TCR activation. The production of IFNγ is thus largely used to identify functional memory CD8⁺ T cells. However, the use of this marker does not discriminate between antigen-induced and cytokine-induced memory CD8⁺ T cells as HP memory phenotype CD8+ T cells produce IFNγ¹⁴. Moreover, the determination of IFNγ levels needs in vitro cell restimulation. On the other hand, CCL5 is a specific biomarker of antigen-induced memory CD8⁺ T cells which does not require in vitro cell restimulation to be investigated. The fact that CCL5 expression is specific of antigen-induced memory cells allows monitoring more closely the generation of memory CD8⁺ T cells in response to an infection or vaccination.

To analyse CCL5 protein expression it is necessary to perform an intracellular staining. This requires a constraining step of cell fixation/permeabilization. However for a routine use, use in clinic or functional analyses after cell sorting, it could be of interest to find a biomarker of antigen-induced memory CD8⁺ T cells that is expressed at the cell surface. Preliminary results obtained in the team show that the expression of the co-stimulatory receptor NKG2D correlates with CCL5 protein expression in memory CD8⁺ T cells. Although this observation will have to be confirmed, this suggests that NKG2D could be another specific biomarker of antigen-induced memory CD8⁺ T cells.

The diversity within the memory CD8⁺ compartment highlights the importance to identify biomarkers to discriminate between memory CD8⁺ subsets. Here, we demonstrated that CCL5 allows discrimination between antigen-induced and cytokine-induced memory CD8⁺ T cells. This discovery has important applications. Indeed, to enumerate all infection-induced or vaccine-induced memory CD8⁺ T cells, it is necessary to know the full spectrum of antigenic peptides harbored by the pathogen or the vaccine. The antigenic peptides can be cultured with the whole population of CD8⁺ T cells and the one that are antigen specific can be detected as they will produce IFNγ. This strategy is time consuming, costly and very often the full spectrum of antigenic is not known. In this study we show that CD8⁺ T cell response to infection can be analysed directly ex vivo in the absence of a priori knowledge of their antigen specificity.

### EXAMPLE 2

Memory CD8 T cells are key players of the immune system, specialized in the clearance of intracellular pathogens such as viruses. These memory cells are generated following a primary infection, through the recognition of antigenic peptides by antigen-specific naive CD8 T cells. They differ from naïve CD8 T cells by their surface phenotype, effector functions and homing pattern. A number of experimental evidence suggest that the pool of memory-phenotype CD8 T cells is much more heterogeneous than initially thought. Indeed, in addition to conventional antigen-induced memory CD8 T cells, sustained γc cytokines stimulation can drive naïve CD8 T cells to differentiate in cytokine-induced memory-phenotype CD8 T cells. These two kinds of memory cells coexist in a normal host and exhibit very similar phenotypic features, making it difficult to distinguish them. A marker specific for one of these subsets would allow the characterization of naturally occurring memory-phenotype CD8 T cells or the quantification of experimentally induced polyclonal antigen specific memory responses. We, therefore, searched for a biomarker enabling the discrimination between these two memory-phenotype CD8 T cell subsets. We identified the chemokine CCL5 and the co-stimulatory receptor NKG2D as new biomarkers that are expressed by antigen-induced memory CD8 T cells generated in response to pathogens or tumors. Indeed, using C57bl/6 mice, we demonstrated that viral or bacterial infection only generates an increase in CCL5+/NKG2D+ memory CD8 T cells. Using different re-stimulation assays, we demonstrated that only NKG2D+ memory CD8 T cells produce effector cytokines in response to antigenic stimulation in vitro. Similar results were obtained, in vivo, following transfer of NKG2D positive or negative memory-phenotype CD8 subsets and re-challenge by the same virus that was used to generate the memory response. Finally, a genomic comparison of the TCR repertoire of NKG2D- and NKG2D+ memory-phenotype CD8 T cells revealed that the latest exhibit a more restricted repertoire, confirming an antigen-driven clonal selection.

### REFERENCES

Throughout this application, various references describe the state of the art to which this invention pertains. The disclosures of these references are hereby incorporated by reference into the present disclosure.
1. Obar, J. J. & Lefrancois, L. Memory CD8+ T cell differentiation Ann N Y Acad Sci 1183, 251-66 (2009).
2. Tan, J. T. & Surh, C. D. T cell memory. Curr Top Microbiol Immunol 311, 85-115 (2006).
3. Northrop, J. K., Wells, A. D. & Shen, H. Cutting edge: chromatin remodeling as a molecular basis for the enhanced functionality of memory CD8 T cells. J Immunol 181, 865-8 (2008).
4. Pihlgren, M., Dubois, P. M., Tomkowiak, M., Sjogren, T. & Marvel, J. Resting memory CD8+ T cells are hyperreactive to antigenic challenge in vitro. J Exp Med 184, 2141-51 (1996).
5. Masopust, D., Vezys, V., Marzo, A. L. & Lefrancois, L. Preferential localization of effector memory cells in nonlymphoid tissue. Science 291, 2413-7 (2001).
6. Mbitikon-Kobo, F. M. et al. Characterization of a CD44/CD122int memory CD8 T cell subset generated under sterile inflammatory conditions. J Immunol 182, 3846-54 (2009).
7. Lee, Y. J., Jameson, S. C. & Hogquist, K. A. Alternative memory in the CD8 T cell lineage. Trends Immuno132, 50-6 (2011).
8. Sprent, J. & Surh, C. D. Normal T cell homeostasis: the conversion of naive cells into memory-phenotype cells. Nat Immunol 12, 478-84 (2011).
9. Goldrath, A. W. & Bevan, M. J. Low-affinity ligands for the TCR drive proliferation of mature CD8+ T cells in lymphopenic hosts. Immunity 11, 183-90 (1999).
10. Kieper, W. C. & Jameson, S. C. Homeostatic expansion and phenotypic conversion of naive T cells in response to self peptide/MHC ligands. Proc Natl Acad Sci USA 96, 13306-11 (1999).
11. Boyman, O., Letourneau, S., Krieg, C. & Sprent, J. Homeostatic proliferation and survival of naive and memory T cells. Eur J Immunol 39, 2088-94 (2009).
12. Kieper, W. C. et al. Overexpression of interleukin (IL)-7 leads to IL-15-independent generation of memory phenotype CD8+ T cells. J Exp Med 195, 1533-9 (2002).
13. Goldrath, A. W., Luckey, C. J., Park, R., Benoist, C. & Mathis, D. The molecular program induced in T cells undergoing homeostatic proliferation. Proc Natl Acad Sci USA 101, 16885-90 (2004).
14. Ge, Q., Hu, H., Eisen, H. N. & Chen, J. Different contributions of thymopoiesis and homeostasis-driven proliferation to the reconstitution of naive and memory T cell compartments. Proc Natl Acad Sci USA 99, 2989-94 (2002).
15. Appay, V. & Rowland-Jones, S. L. RANTES: a versatile and controversial chemokine. Trends Immunol 22, 83-7 (2001).
16. Bacon, K. B., Premack, B. A., Gardner, P. & Schall, T. J. Activation of dual T cell signaling pathways by the chemokine RANTES. Science 269, 1727-30 (1995).
17. Bodaghi, B. et al. Chemokine sequestration by viral chemoreceptors as a novel viral escape strategy: withdrawal of chemokines from the environment of cytomegalovirus-infected cells. J Exp Med 188, 855-66 (1998).
18. Mamalaki, C. et al. Positive and negative selection in transgenic mice expressing a T-cell receptor specific for influenza nucleoprotein and endogenous superantigen. Dev Immunol 3, 159-74 (1993).
19. Arpin, C. et al. Hyperproliferative response of a monoclonal memory CD8 T cell population is characterized by an increased frequency of clonogenic precursors. J Immunol 168, 2147-53 (2002).
20. Marcais, A. et al. Maintenance of CCL5 mRNA stores by post-effector and memory CD8 T cells is dependent on transcription and is coupled to increased mRNA stability. Eur J Immunol 36, 2745-54 (2006).
21. Swanson, B. J., Murakami, M., Mitchell, T. C., Kappler, J. & Marrack, P. RANTES production by memory phenotype T cells is controlled by a posttranscriptional, TCR-dependent process. Immunity 17, 605-15 (2002).
22. Walzer, T. et al. Cutting edge: immediate RANTES secretion by resting memory CD8 T cells following antigenic stimulation. J Immunol 170, 1615-9 (2003).
23. Haluszczak, C. et al. The antigen-specific CD8+ T cell repertoire in unimmunized mice includes memory phenotype cells bearing markers of homeostatic expansion. J Exp Med 206, 435-48 (2009).

1. A method for determining the presence of a population of antigen-induced memory CD8+ T cells in a sample, comprising
i) isolating a population of CD8+ T cells from the sample
ii) detecting in said population expression of CCL5 or NKG2D and
iii) concluding that at least one population of said antigen-induced memory CD8+ T cells is present in a said sample when CCL5 or NKG2D is detected as in step ii).

2. The method according to claim 1 wherein the sample is a murine sample or a human sample.

3. The method according to claim 1 wherein the expression of CCL5 or NKG2D at step ii) is detected at the protein level or at the mRNA level.

4. The method according to claim 1, further comprising a step of determining whether the population of antigen-induced memory CD8+ T cells is specific for an antigen.

5. The method according to claim 1, further comprising a step of isolating the population of antigen-induced memory CD8+ T cells detected at step iii).

6. The method according to claim 1, wherein the antigen is selected from the group consisting of antigens associated with a pathogenic organism, tumor associated antigens and allergens.

7. A method for determining whether a subject has developed a memory T cell response in a context of a disorder selected from the group consisting of infections with a pathogenic organism, cancers, allergies, and autoimmune disorders, comprising
i) isolating a population of CD8+ T cells from said subject,
ii) detecting in said population expression of CCL5 or NKG2D, and
iii) concluding that said subject has developed a memory T cell response if CCL5 or NKG2D is detected in at least one population of said CD8+ T cells.

8. A method for determining the efficacy of a treatment for a disorder in a subject, wherein said disorder is selected from the group consisting of infections with a pathogenic organism, cancers, allergies, and autoimmune disorders, comprising
i) treating said subject for said disorder,
ii) isolating a population of CD8+ T cells from said subject,
iii) detecting expression of CCL5 or NKG2D in said population of CD8+ T cells, and
iv) concluding that said subject has developed a memory T cell response and thus that said treatment is efficacious if CCL5 or NKG2D is detected in said population of said CD8+ T cells.

9. A method for testing efficacy of a vaccine
i) vaccinating a subject
ii) isolating a population of CD8+ T cells from said vaccinated subject
iii) detecting expression of CCL5 or NKG2D in said population of CD8+ T cells and
iv) concluding that said vaccinated subject has developed a memory T cell response and thus that said vaccine is efficacious if CCL5 or NKG2D is detected in at least one population of said CD8+ T cells.

10. The method according to claim 9 further comprising the steps of
i) administering a placebo to a control subject;
ii) determining, in samples from said vaccinated subject and said control subject, a level of antigen-induced memory CD8+ T cells specific for an antigen of a said vaccine; and
iii) if said level of antigen-induced memory CD8+ T cells specific for an antigen is higher for said vaccinated subject than for said control subject, then
iv) concluding that said vaccine was efficient.

11. The method according to claim 9 further comprising a step of comparing the level of CCL5 or NKG2D containing, antigen-induced memory CD8+ T cells before and after administration of the vaccine to the subject, wherein an increase in the level of the CCL5 or NKG2D containing, antigen-induced memory CD8+ T cells after administration indicates that the vaccine preparation was efficient.

12. The method according to claim 9 further comprising the steps of
i) determining the levels of the whole population of antigen-induced memory CD8+ T cells before and after administration of the vaccine to the subject and
ii) comparing the levels of the whole population of antigen-induced memory CD8+ T cells determined before and after administration, wherein an increase in the level of the whole population of antigen-induced memory CD8+ T cells after administration indicates that the vaccine preparation was efficient.

13. A method for screening a combination of antigens and/or immunoadjuvants for the preparation of a vaccine preparation, comprising
i) administering said combination of antigens and/or immunoadjuvants to a subject,
ii) isolating a population of CD8+ T cells from said subject,
iii) detecting expression of CCL5 or NKG2D in said population of CD8+ T cells, and
iv) concluding that said subject has developed a memory T cell response and thus that said combination of antigens and/or immunoadjuvants is efficacious and suitable for use in a vaccine if CCL5 or NKG2D is detected in at least one population of said CD8+ T cells.
